(19) United States
(12) Patent Application Publication
(10) Pub. No.: US 2025/0090192 A1
(43) Pub. Date: Mar. 20, 2025

(54) TREATMENT TOOL

(71) Applicant: OLYMPUS MEDICAL SYSTEMS CORP., Tokyo (JP)

(72) Inventor: Keisuke NAGAO, Tokyo (JP)

(21) Appl. No.: 18/884,863

(22) Filed: Sep. 13, 2024

Related U.S. Application Data
(60) Provisional application No. 63/583,559, filed on Sep. 18, 2023.

(51) Int. Cl. A61B 17/32; A61B 17/00; A61B 17/29; A61B 17/295

(52) U.S. Cl. CPC A61B 17/320092; A61B 17/295; A61B 2017/00367; A61B 2017/00477; A61B 2017/2901; A61B 2017/2919; A61B 2017/2925; A61B 2017/2932; A61B 2017/320094

(57) ABSTRACT

A treatment tool can include a first gripping piece and a second gripping piece that is openable and closable relatively to the first gripping piece. A longitudinal portion can be configured to transmit driving force to open or close the first gripping piece and the second gripping piece. The treatment tool can further include housing that has a stationary handle and to which the longitudinal portion is connected and a movable handle rotatable about a first rotation axis in the housing. The tool can also include a transmitter that is movable in the housing and configured to transmit force generated by a grip on the movable handle to the longitudinal portion and a link that has one end pivotally supported to be rotatable about a second rotation axis in a connector of the movable handle and another end pivotally supported to be rotatable about a third rotation axis in the transmitter.

### CROSS-REFERENCE TO RELATED APPLICATION

This application is based on and claims priority under 35 U.S.C. § 119 to U.S. Provisional Application No. 63/583,559, filed Sep. 18, 2023, the entire contents of which are incorporated herein by reference.

### TECHNICAL FIELD

The present disclosure relates to treatment tools.

### BACKGROUND

A treatment tool can apply treatment energy to a site to be treated in body tissue (hereinafter, referred to as a target site) to treat the target site (see, for example, Japanese Patent Application Laid-open No. 2011-235117). In some examples a link connected to one end of a trigger can be included, the trigger functioning as a movable handle. The link can include a driver connected to the other end of the link. A user can manipulate the trigger and the link thereby causes the driver to move forward and a jaw is thereby operated. The driver can be attached to the jaw and causing the jaw to operate.

### SUMMARY

In some embodiments, a treatment tool according to the disclosure includes: a first gripping piece and a second gripping piece that is openable and closable relatively to the first gripping piece. The second gripping piece can be configured to grip body tissue between the first gripping piece and the second gripping piece. A longitudinal portion with a distal end and a proximal end can be formed along a longitudinal axis. The longitudinal portion can be configured to transmit driving force to open or close the first gripping piece and the second gripping piece that are provided at the distal end.

The treatment tool can further include a housing that has a stationary handle and to which the proximal end of the longitudinal portion is connected and a movable handle that is pivotally supported to be rotatable about a first rotation axis in the housing. The movable handle can include a connector extending from the first rotation axis toward the distal end and the movable handle can be configured to move in a direction toward or away from the stationary handle.

The treatment tool can also include a transmitter configured to be movable along the longitudinal axis in the housing and that is capable of transmitting force generated by a grip on the movable handle to the longitudinal portion. A link that has one end pivotally supported to be rotatable about a second rotation axis in the connector and another end pivotally supported to be rotatable about a third rotation axis in the transmitter can be included. The link can be coupled to each of the movable handle and the transmitter. When the movable handle moves in the direction toward the stationary handle according to operation on the movable handle by an operator, the link is configured to move the transmitter along the longitudinal axis toward the distal end with the first rotation axis being a fulcrum.

The above and other features, advantages and technical and industrial significance of this disclosure will be better understood by reading the following detailed description of embodiments of the disclosure, when considered in connection with the accompanying drawings.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a diagram illustrating a schematic configuration of a treatment tool according to an embodiment.

FIG. 2 illustrates an example of a diagram illustrating a configuration of the treatment tool according to an embodiment.

FIG. 3 illustrates an example of a diagram illustrating the configuration of the treatment tool according to an embodiment.

FIG. 4 illustrates an example of a diagram illustrating the configuration of the treatment tool according to an embodiment.

FIG. 5 illustrates an example of a diagram illustrating the configuration of the treatment tool according to an embodiment.

FIG. 6 illustrates an example of a diagram illustrating the configuration of the treatment tool according to an embodiment.

FIG. 7 illustrates an example of a diagram illustrating the configuration of the treatment tool according to an embodiment.

FIG. 8 illustrates an example of an enlarged view of a connector of a movable handle included in the treatment tool according to an embodiment.

FIG. 9 illustrates an example of an enlarged view of a state where a link has been coupled to the connector of the movable handle included in the treatment tool according to an embodiment.

FIG. 10 illustrates an example of a diagram of a schematic configuration of the link included in the treatment tool according to an embodiment.

FIG. 11 illustrates an example of a diagram of a schematic configuration representing a configuration of a transmitter included in the treatment tool according to the embodiment;

FIG. 12 illustrates an example of a diagram illustrating a state where the link and the movable handle have been coupled to the transmitter included in the treatment tool according to an embodiment.

FIG. 13 illustrates an example of a diagram illustrating a state where the link, the movable handle, and an opening and closing mechanism have been coupled to the transmitter included in the treatment tool according to the embodiment.

FIG. 14 illustrates an example of a diagram schematically illustrating rotation force by the movable handle included in the treatment tool according to the embodiment.

### DETAILED DESCRIPTION

A mode for implementing the present disclosure (hereinafter, an embodiment or an example) will be described hereinafter while reference is made to the drawings. The present disclosure is not to be limited by the embodiment(s) or example(s) described hereinafter. Furthermore, the drawings referred to in the following description schematically illustrate shapes, sizes, and positional relations merely to an extent that allows the present disclosure to be understood. That is, the present disclosure is not to be limited only to the shapes, sizes, and positional relations exemplified by the drawings. Any portions that are the same will be assigned with the same reference sign throughout the drawings.

### Schematic Configuration of Treatment Tool

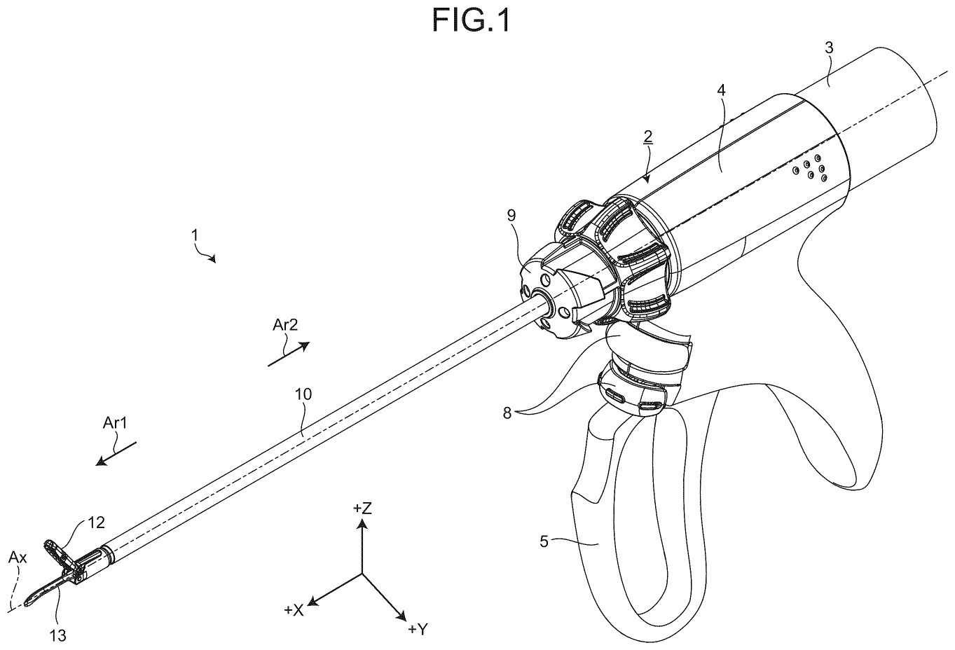

FIG. 1 illustrates an example of a diagram illustrating a schematic configuration of a treatment tool according to an embodiment of the present disclosure. A treatment tool 1 illustrated in FIG. 1 can apply ultrasound energy and high frequency energy to a site to be treated in body tissue (hereinafter, referred to as a “target site”) to thereby treat the target site. This treatment can be, for example, coagulation (sealing) and incision of the target site. Furthermore, the coagulation and the incision can be performed simultaneously with the treatment. As illustrated in FIG. 1, the treatment tool 1 can include a handpiece 2 and an ultrasound transducer 3.

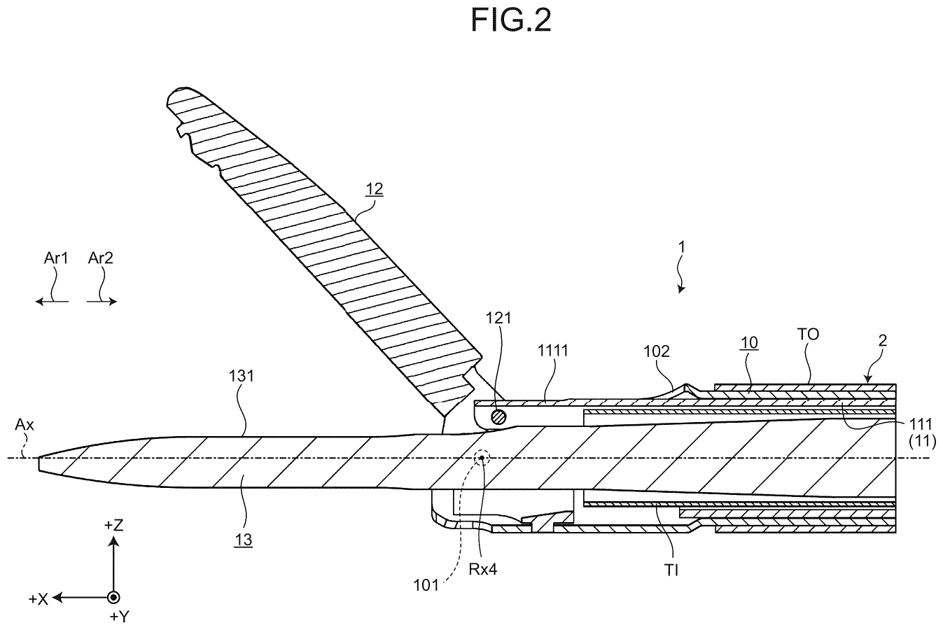

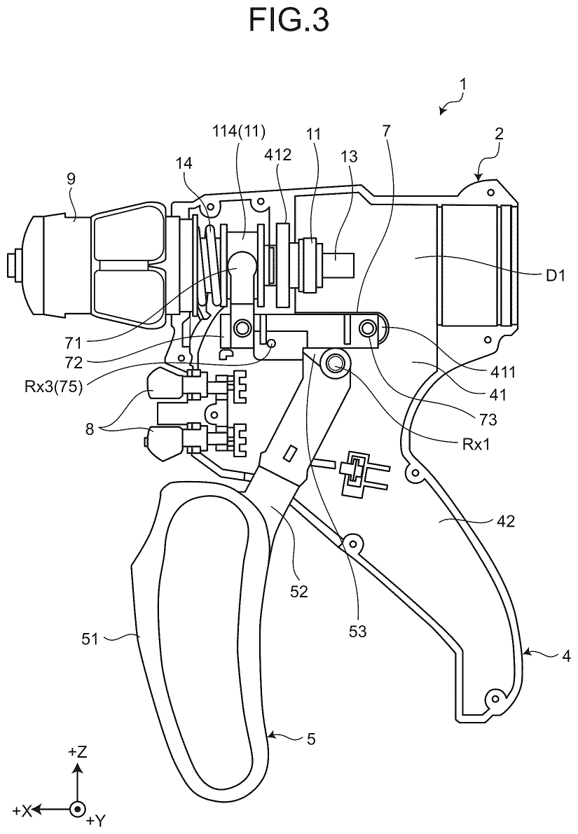

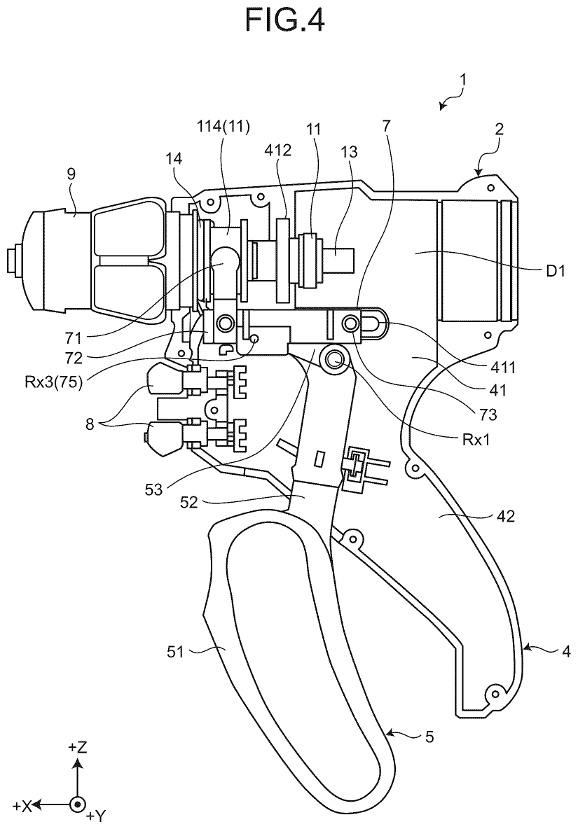

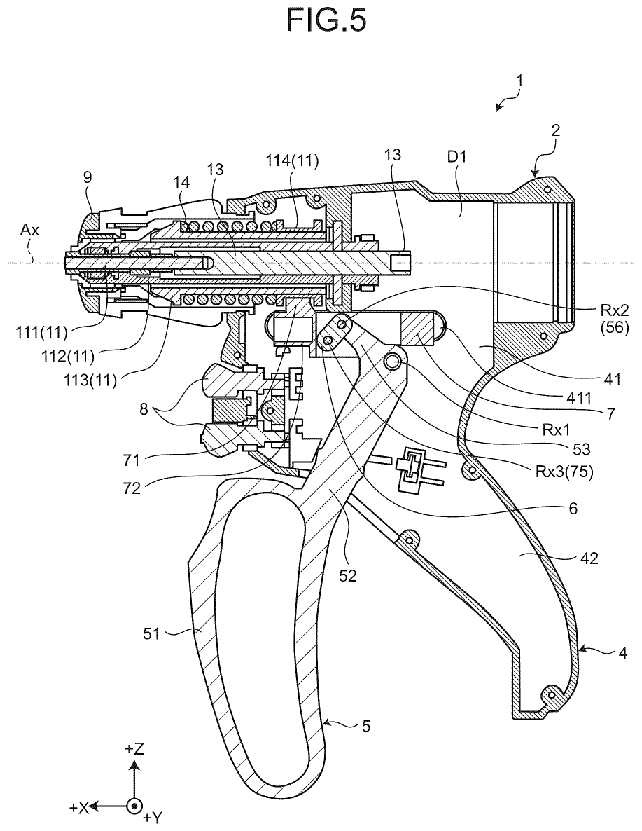

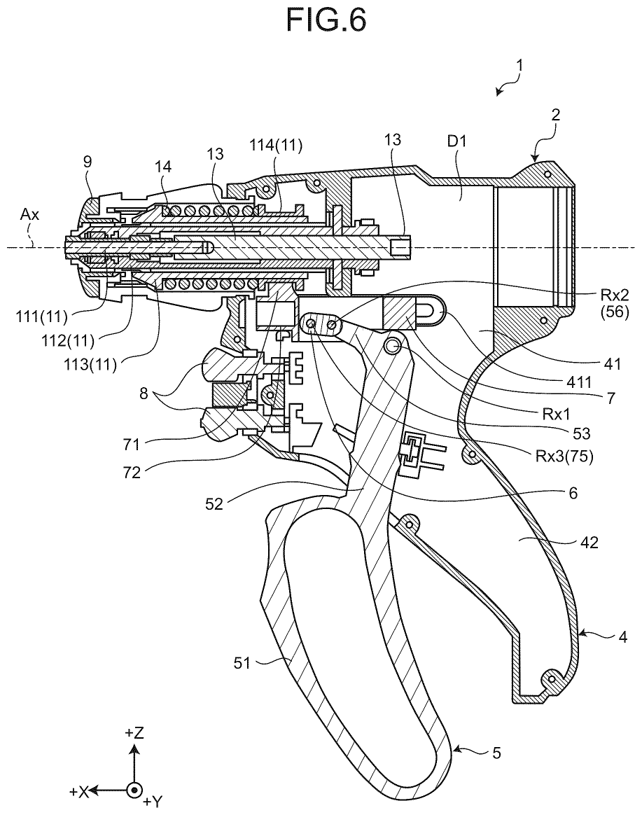

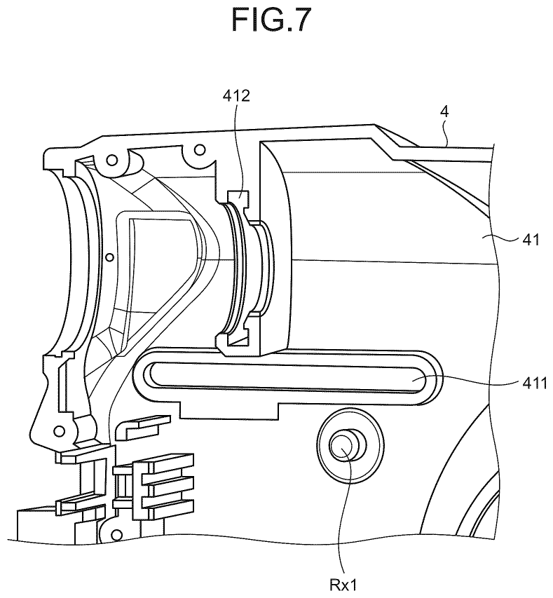

FIG. 2 to FIG. 7 are example diagrams illustrating a configuration of the treatment tool 1. Specifically, FIG. 2 illustrates an example of a sectional view of the treatment tool 1 cut along an X-Z plane including a central axis Ax and viewed from a positive direction along a Y-axis. FIG. 3 and FIG. 4 illustrate example side views of the treatment tool 1, from which part of its housing 4 has been removed, in a state where the ultrasound transducer 3 has been removed from the handpiece 2. FIG. 5 and FIG. 6 illustrate example sectional views of the treatment tool 1 cut along the X-Z plane including the central axis Ax and viewed from the positive direction along the Y-axis, in the state where the ultrasound transducer 3 has been removed from the handpiece 2. FIG. 7 illustrates an example of a side view of main parts of the housing of the treatment tool 1. In description of the configuration of the treatment tool 1, X, Y, and Z coordinate axes that are an X-axis, a Y-axis, and a Z-axis, which are orthogonal to one another, will be used hereinafter. As illustrated, the X-axis is an axis parallel to the central axis Ax (FIG. 1) of a shaft 10 described later. The Y-axis is an axis orthogonal to the X-axis in FIG. 1. The Z-axis is an axis along a vertical direction in FIG. 1. Furthermore, one direction along the central axis Ax (a positive direction along the X-axis) will hereinafter be referred to as a distal direction Ar1 and the other direction along the central axis Ax (a negative direction along the X-axis) will hereinafter be referred to as a proximal direction Ar2.

The handpiece 2 can include, as illustrated in FIG. 1 to FIG. 7, the housing 4 (FIG. 1 and FIG. 2 to FIG. 7), a movable handle 5 (FIG. 1 and FIG. 2 to FIG. 6), a link 6 (FIG. 5 and FIG. 6), a transmitter 7 (FIG. 3 to FIG. 6), a rotation knob 9 (FIG. 1 and FIG. 3 to FIG. 6), switches 8 (FIG. 1 and FIG. 2 to FIG. 6), the shaft 10 (FIG. 1 and FIG. 2), an opening and closing mechanism 11 (FIG. 3 to FIG. 6), a jaw (first jaw member) 12 (FIG. 1 and FIG. 2), a vibration transmitter (second jaw member) 13 (FIG. 1 to FIG. 6), and a biasing portion 14 (FIG. 3 to FIG. 6).

The housing 4 can support the whole treatment tool 1. The housing 4 can include: a case body 41 located coaxial with the central axis Ax and approximately cylindrical and a stationary handle 42 extending in a negative direction along the Z-axis from the case body 41 and is gripped by an operator, such as an operating surgeon.

The case body 41 can include a groove portion 411 (FIG. 7) where the transmitter 7 can be inserted and that is for the transmitter 7 to be fitted in and be movable forward and backward according to opening and closing operation of the movable handle 5. The case body can further include a holding unit 412 (FIG. 7) that holds the opening and closing mechanism 11 and a first rotation axis Rx1 (fulcrum) pivotally supporting the movable handle 5.

The movable handle 5 can receive each of closing operation and opening operation by an operator, such as an operating surgeon. The closing operation can include gripping the movable handle 5 with the fingers while placing the palm on the stationary handle 42. Furthermore, the opening operation can include operation of releasing force of the gripping.

The movable handle 5 can be positioned inside the housing 4. The movable handle 5 can be pivotally supported to be rotatable about the first rotation axis Rx1 (fulcrum) parallel to the Y-axis. The movable handle 5 can include a gripped portion 51, a handle base portion 52, and a connector 53.

The gripped portion 51 can be a portion that receives each of the closing operation and the opening operation by an operator and can be positioned outside the housing 4.

The handle base portion 52 can extend from an upper end of the gripped portion 51 and can be pivotally supported to be rotatable about the first rotation axis Rx1 (see FIG. 3 to FIG. 6). An end portion of the handle base portion 52 can include the connector 53 formed therein. The end portion can be located or oriented in a positive direction along the Z-axis. The connector 53 can extend from the first rotation axis Rx1 (fulcrum) toward a distal end.

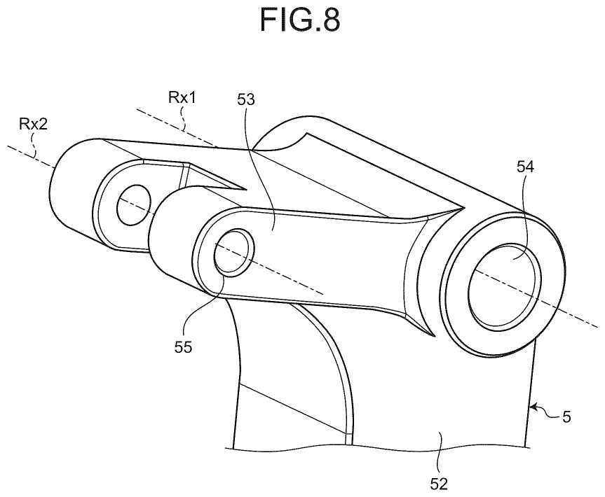

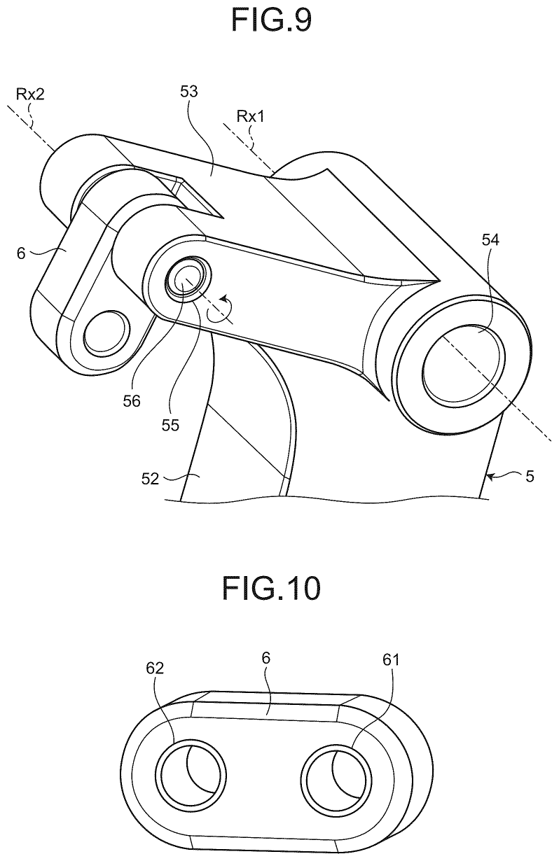

FIG. 8 illustrates an example of an enlarged view of the connector 53 of the movable handle 5. FIG. 9 illustrates an example of an enlarged view of a state where the link 6 has been coupled to the connector 53 of the movable handle 5.

The connector 53 can be formed to extend from the first rotation axis Rx1 (fulcrum) toward the distal end and to protrude in the positive direction along the X-axis in a state of being divided in two branches, as illustrated in FIG. 8 and FIG. 9. The connector 53 can include a hole portion 54 at a proximal end thereof and a hole portion 55 at a distal end thereof. The connector 53 can be pivotally supported to be rotatable about the first rotation axis Rx1, with the first rotation axis Rx1 inserted in the hole portion 54, the first rotation axis Rx1 having been provided in the case body 41. Furthermore, the connector 53 can pivotally support the link 6 such that the link 6 is rotatable about a second rotation axis Rx2, by insertion of a pin 56 in the hole portion 55 in a state where the link 6 has been accommodated therein (FIG. 9). Furthermore, the second rotation axis Rx2 can be arranged more upward than the first rotation axis Rx1 in the connector 53 (see FIG. 3 to FIG. 6, FIG. 8, and FIG. 9).

The link 6 can be connected to each of the movable handle 5 and the transmitter 7. Specifically, the link 6 can include a proximal end pivotally supported to be rotatable about the second rotation axis Rx2 in the connector 53 and a distal end pivotally supported to be rotatable about a third rotation axis Rx3 in the transmitter 7 (see FIG. 5 and FIG. 6). Furthermore, the link 6 can be arranged between the central axis Ax along a longitudinal axis of an inner pipe 111 in the housing 4 and the first rotation axis Rx1. Specifically, in a case where a direction, in which the stationary handle 42 extends with respect to the housing 4, is downward (the negative direction along the X-axis), the link 6 can be arranged more upward (in the positive direction along the X-axis) than the first rotation axis Rx1 and in the housing 4.

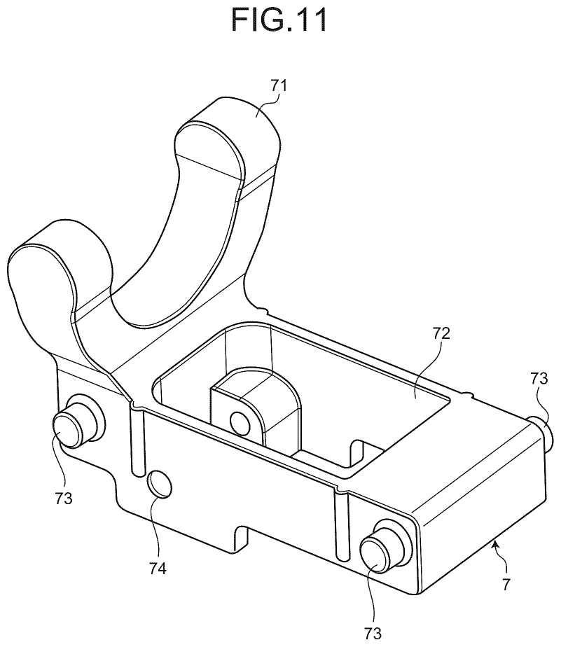

FIG. 10 illustrates an example of a diagram of a schematic configuration of the link 6. As illustrated in FIG. 10, the link 6 can have or be formed in an oval shape with a thickness and can include a hole portion 61 and a hole portion 62. The link 6 can be coupled to the connector 53, with the pin 56 inserted in the hole portion 61, the pin 56 functioning as the second rotation axis Rx2, and the link 6 can be pivotally supported to be rotatable about the second rotation axis Rx2 (see FIG. 9). Furthermore, the link 6 can be coupled to the transmitter 7, with a pin 75 inserted in the hole portion 62, the pin 75 functioning as the third rotation axis Rx3, and the link 6 is pivotally supported to be rotatable about the third rotation axis Rx3 (see FIG. 5 and FIG. 6).

The transmitter 7 can be movable along the longitudinal axis in the housing 4 and can transmit a force generated by a grip on the movable handle 5 to the inner pipe 111 of the opening and closing mechanism 11 described later. Furthermore, the transmitter 7 can be engaged with a slider 114 of the opening and closing mechanism 11 described later.

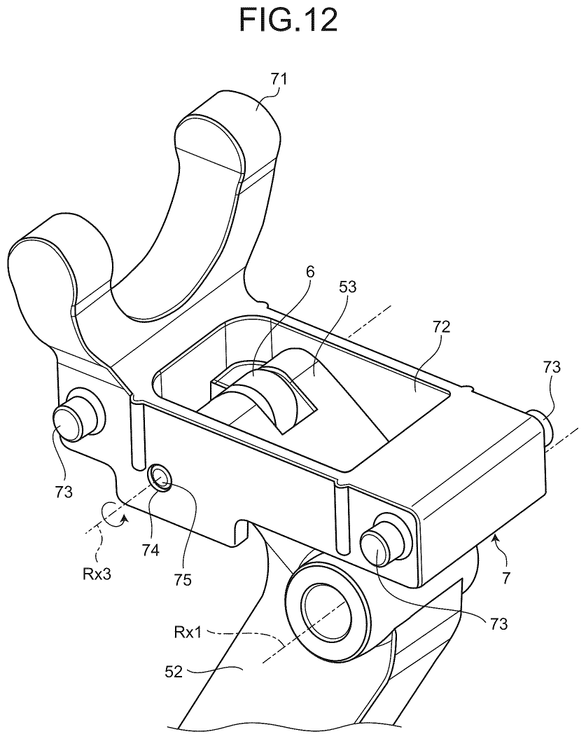

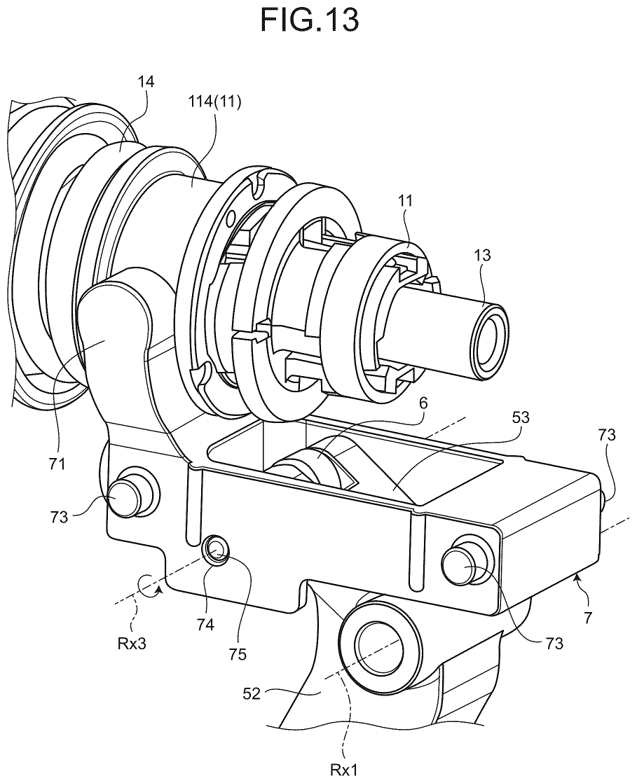

FIG. 11 illustrates an example of a schematic diagram illustrating a configuration of the transmitter 7. FIG. 12 is an example of a diagram illustrating a state where the link 6 and the movable handle 5 have been coupled to the transmitter 7. FIG. 13 illustrates an example of a diagram illustrating a state where the link 6, the movable handle 5, and the opening and closing mechanism 11 have been coupled to the transmitter 7.

As illustrated in FIG. 11 to FIG. 13, the transmitter 7 can include an engagement portion 71 that can engaged with the slider 114 included in the opening and closing mechanism 11. The transmitter 7 can further include an accommodating portion 72 that can accommodate the link 6 and the connector 53 therein, projection portions 73 provided at the front and back of lateral surfaces of the accommodating portion 72 and that each have a cylindrical (or substantially cylindrical) shape, and a hole portion 74. The link 6 can be pivotally supported to be rotatable about the third rotation axis Rx, by insertion of the pin 75 in the hole portion 74 of the transmitter 7 in a state where the link and the connector 53 of the movable handle 5 have been stored in the accommodating portion 72. The projection portions 73 can be inserted in the groove portion 411 of the case body 41 of the housing 4, and move forward and backward in the groove portion 411 along a longitudinal axis of the shaft 10, according to operation of the link 6 and the movable handle 5.

The switches 8 can be provided, as illustrated in FIG. 1 and FIG. 3 to FIG. 6, in a state of being exposed outside from a lateral surface of the housing 4, the lateral surface being in the distal direction Ar1. The switches 8 can receive treatment operation by an operator. The treatment operation includes operation to apply treatment energy to a target site.

The rotation knob 9 can have or be formed in an approximately cylindrical shape coaxial with the central axis Ax and can be provided, as illustrated in FIG. 1 and FIG. 3 to FIG. 6, near an end of the housing 4, the end being in the distal direction Ar1. The rotation knob 9 can receive rotation operation from an operator. The rotation operation can rotate the rotation knob 9 about the central axis Ax, relatively to the housing 4. Furthermore, the rotation of the rotation knob 9 can rotate the jaw 12 and the vibration transmitter 13 about the central axis Ax.

The shaft 10 can be a cylindrical pipe formed of a material, such as metal. An outer peripheral surface of the shaft 10 can be covered with an outer tube TO (FIG. 2) that can be electrically insulating. Furthermore, a pin 101 (FIG. 2) that extends in a direction orthogonal to the plane of paper of FIG. 1 and FIG. 2 and pivotally supports the jaw 12 such that the jaw 12 can be rotatable about a fourth rotation axis Rx4 (FIG. 2) can be fixed to an end portion of the shaft 10, the end portion being in the distal direction Ar1. Furthermore, in the positive direction along the Z-axis, a notched portion 102 can be formed in the end portion of the shaft 10, the end portion being in the distal direction Ar1, the notched portion 102 extending from a distal end of the shaft 10 in the proximal direction Ar2.

The opening and closing mechanism 11 can be configured as a mechanism that opens and closes the jaw 12 relatively to an end portion 131 (hereinafter, referred to as a treatment portion 131) of the vibration transmitter 13, the end portion 131 being in the distal direction Ar1, according to opening operation and closing operation on the movable handle by an operator. This opening and closing mechanism 11 can include the inner pipe 111 (FIG. 2), a holder 112 (FIG. 5 and FIG. 6), a slider receiver (slider receiving member) 113 (FIG. 5 and FIG. 6), and the slider 114 (FIG. 3 to FIG. 6).

The inner pipe 111 can be a cylindrical pipe having a diameter with a dimension smaller than that of the shaft 10. Furthermore, the inner pipe 111 can be inserted in the shaft 10 in a state of being coaxial with the shaft 10. An arm portion 1111 that protrudes in the distal direction Ar1 can be provided, as illustrated in FIG. 2, in the inner pipe 111, the arm portion 1111 being in the positive direction along the Z-axis at an end portion of the inner pipe 111, the end portion being in the distal direction Ar1. A pin 121 can be provided in the jaw 12 and extending in a state of being parallel to the fourth rotation axis Rx4 (the pin 101) can be inserted in the arm portion 1111. The inner pipe 111 can correspond to a longitudinal portion according to the present disclosure.

The holder 112 can be formed of a material that is electrically insulating, such as resin, and can have or be formed in an approximately cylindrical shape. As illustrated in FIG. 5 and FIG. 6, the holder 112 can be inserted in the rotation knob 9 and the housing 4, in a state of straddling the rotation knob 9 and the housing 4. The holder 112 can hold the vibration transmitter 13 that has been inserted in the holder 112. Furthermore, the holder 112 can be mechanically connected to the rotation knob 9 and the shaft 10, at an end portion of the holder 112, the end portion being in the distal direction Ar1. That is, the holder 112, the shaft 10, the jaw 12, and the vibration transmitter 13 can rotate, together with the rotation knob 9, about the central axis Ax, according to rotation operation on the rotation knob 9 by an operator, such as an operating surgeon.

The slider receiver 113 can be formed of a material that is electrically insulating, such as resin, and can have an approximately cylindrical shape. The slider receiver 113 can be provided movably along the central axis Ax relatively to the holder 112, in a state where the holder 112 has been inserted in the slider receiver 113. An end portion of the slider receiver 113, the end portion being in the distal direction Ar1, can be connected to an end portion of the inner pipe 111, the end portion being in the proximal direction Ar2, in a state where the end portion of the slider receiver 113 is allowed to move along the central axis Ax relatively to the holder 112 but restricted from rotating about the central axis Ax. That is, the slider receiver 113 and the inner pipe 111 can rotate, together with the rotation knob 9, about the central axis Ax, according to rotation operation on the rotation knob 9 by an operator.

The slider 114 can have or be formed in an approximately cylindrical shape and can be provided movably along the central axis Ax, relatively to the slider receiver 113, in a state where the slider receiver 113 has been inserted in the slider 114. The slider 114 can be engaged with the movable handle 5 via the transmitter 7 and the link 6, as described above.

At least part of the jaw 12 can be formed of an electrically conducting material. The jaw 12 can correspond to a second gripping piece according to the present disclosure.

The vibration transmitter 13 can be formed of an electrically conducting material and can have or be formed in an elongated shape linearly extending along the central axis Ax. The vibration transmitter 13 can be inserted in the inner pipe 111, in a state where the treatment portion 131 protrudes outside the inner pipe 111, as illustrated in FIG. 2. In this state, an end portion of the vibration transmitter 13, the end portion being in the proximal direction Ar2, can be mechanically connected to the ultrasound transducer 3, as illustrated in FIG. 1. That is, the vibration transmitter 13 can transmit ultrasound vibration generated by the ultrasound transducer 3 from the end portion of the vibration transmitter 13 to the treatment portion 131, the end portion being in the proximal direction Ar2. In an embodiment according to the present disclosure, the ultrasound vibration can be longitudinal vibration that is vibration along the central axis Ax. An outer peripheral surface of the vibration transmitter 13 can be covered with an inner tube TI (FIG. 2) that is electrically insulating (e.g., can be formed from or include an electrically insulating material), to provide electric insulation between the shaft 10 and the inner pipe 111 and the vibration transmitter 13. Furthermore, the vibration transmitter 13 can correspond to a first gripping piece according to the present disclosure.

The ultrasound transducer 3 can generate ultrasound vibration, under control by a control device not illustrated in the drawings. Specifically, the ultrasound transducer 3 can generate longitudinal vibration (ultrasound vibration) that includes vibration along the central axis Ax, on the basis of driving electric power supplied from the control device not illustrated in the drawings. The treatment portion 131 (see FIG. 2) can be vibrated at a desired amplitude by the longitudinal vibration. From the treatment portion 131, the ultrasound vibration can be applied to the target site gripped between the jaw 12 and the treatment portion 131. In other words, ultrasound energy can be applied to the target site from the treatment portion 131.

Furthermore, for example, in applying high frequency energy to a target site, a control device can supply high frequency electric power between the jaw 12 and the vibration transmitter 13 through an electric cable. High frequency electric current thereby can flow in the target site gripped between the jaw 12 and the treatment portion 131. In other words, high frequency energy can be applied to the target site.

### Operation of Treatment Tool

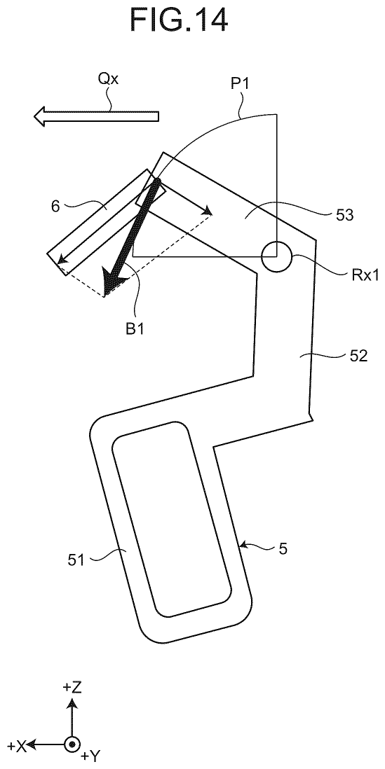

Operation on the movable handle 5 by an operator, such as an operating surgeon, will be described next. FIG. 14 illustrates an example of a diagram schematically illustrating rotation force by the movable handle 5.

As illustrated in FIG. 14, in a case where closing operation is performed on the movable handle 5 by an operator, (from a state in FIG. 3 and FIG. 5 to a state in FIG. 4 and FIG. 6), rotation force P1 (a point of application) can be generated to be directed to the front (to a distal end) of the treatment tool 1. Therefore, the force due to the closing operation by the operator can be transmitted forward (see an arrow Qx) even through the movable handle 5, the link 6, and the transmitter 7. That is, the link 6 can be arranged more upward than the first rotation axis Rx1 in the movable handle 5 of the treatment tool 1, the second rotation axis Rx2 can be at a position higher than that of the first rotation axis Rx1, and force B1 generated upon rotation about the first rotation axis Rx1 can thus also directed forward. In other words, because the link 6 functioning as the point of application is at a higher position than the first rotation axis Rx1 functioning as the fulcrum in the treatment tool 1, a component force along the longitudinal axis of the shaft 10 can match the direction, along which the link 6 has been provided.

As a result, the operator, can move the slider 114 forward by moving the transmitter 7 forward along the groove portion 411 with a small amount of force and a small stroke, and the target site is thus able to be readily gripped by the jaw 12 and the vibration transmitter 13.

Furthermore, in the case where the closing operation is performed on the movable handle 5 of the treatment tool 1 (from the state in FIG. 3 and FIG. 5 to the state in FIG. 4 and FIG. 6), the rotation force P1 can be generated to be directed to the front (to the distal end) of the treatment tool 1, and a space D1 (FIG. 3 to FIG. 6) at the back of the housing 4 is thus able to be increased in size and design freedom for a shape of the ultrasound transducer 3 is thereby able to be increased.

According to the embodiment described above, in a case where the movable handle 5 moves in a direction toward the stationary handle 42 according to operation on the movable handle 5 by an operator, the link 6 can move the transmitter 7 toward the distal end along the longitudinal axis of the shaft 10, with the first rotation axis Rx1 being the fulcrum, power needed in the operation of the movable handle 5 is thus efficiently transmitted, and the amount of that power can thereby be reduced.

Furthermore, according to the embodiment, the link 6 can be arranged between the central axis Ax along the longitudinal axis of the inner pipe 111 in the housing 4 and the first rotation axis Rx1, the force B1 generated upon rotation of the movable handle 5 about the first rotation axis Rx1 can thereby directed forward, and an operator can move the slider 114 forward by moving the transmitter 7 forward along the groove portion 411 with a small amount of force and a small stroke.

Furthermore, according to the embodiment described above, the first rotation axis Rx1 can be provided at a position fixed relatively to the housing 4 and the movable handle 5 is thus able to be readily installed in manufacture.

### Other Embodiments

A mode for implementing the present disclosure has been described thus far, but the present disclosure is not to be limited only to the above described embodiment. A configuration to apply both ultrasound energy and high frequency energy to a target site is adopted in the above described embodiment, but the embodiment is not limited to this configuration. For example, a configuration to be adopted may be a configuration to apply only ultrasound energy to a target site, a configuration to apply only high frequency energy to a target site, a configuration to apply only another kind of energy that is neither ultrasound energy nor high frequency energy to a target site, or a configuration including any combination of these configurations.

Furthermore, in an embodiment, inner pipe driving is adopted, but without being limited to the inner pipe driving, for example, outer pipe driving may be applied to the embodiment.

According to the present disclosure, an effect of enabling reduction of force needed in operation of a movable handle and improvement of operability thereof can be achieved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A treatment tool, comprising:
a first jaw;
a second jaw configured to open and close relative to the first jaw;
a shaft extending along a longitudinal axis;
a first handle located proximally relative to the shaft;
a second handle pivotally supported about a first axis relative to the first handle, wherein the second handle includes a connector extending from the first axis toward a distal end of the shaft;
a transmitter configured to move along the longitudinal axis and configured to transmit force generated by moving the second handle; and
a link pivotally supported about a second axis relative to the connector and pivotally supported about a third axis relative to the transmitter,
wherein when the second handle moves toward the first handle, the link is configured to move the transmitter along the longitudinal axis toward the distal end of the shaft.

2. The treatment tool according to claim 1, further comprising:
a housing connected to a proximal end of the shaft and including the first handle, wherein the link is arranged between a central axis along the longitudinal axis in the housing and the first axis.

3. The treatment tool according to claim 1, further comprising:
a housing connected to a proximal end of the shaft and including the first handle, wherein the first axis is fixed relatively to the housing.

4. The treatment tool according to claim 1, further comprising:
a holder configured to hold the shaft;
a slider receiving body receiving the holder and configured to move relative to the holder along a central axis along the longitudinal axis; and
a slider into receiving the slider receiving body and configured to move relative to the slider receiving body, wherein the transmitter is engaged with the slider.

5. The treatment tool according to claim 4, wherein the transmitter couples the link and the slider, the link and the slider are located distally relative to the first axis.

6. The treatment tool according to claim 1, wherein the shaft is an inner pipe.

7. The treatment tool according to claim 1, wherein the second jaw is configured to grip body tissue between the first jaw and the second jaw.

8. The treatment tool according to claim 1, wherein the treatment tool is configured to apply ultrasound energy to body tissue to treat the body tissue.

9. The treatment system to claim 1, wherein
the connector comprises two branches extending toward the distal end of the shaft.

10. The treatment system of claim 1, wherein the connector comprises a hole defining the second axis.

11. The treatment system of claim 1, further comprising:
a pin inserted into a first hole of the connector and a second hole of the link.

12. The treatment system of claim 9, wherein the link comprises:
a first hole defining the second axis; and
a second hole defining the third axis.

13. The treatment system of claim 9, wherein a first distance between the link and the longitudinal axis is smaller than a second distance between the first axis and the longitudinal axis

14. The treatment system of claim 13, wherein the link is located distally relative to the first axis and located between the shaft and the first axis.

15. A treatment system, comprising:
an elongate shaft extending along a longitudinal axis;
a first jaw member and a second jaw member at a distal end of the elongate shaft, wherein at least one of the first and second jaw members is movable relative to the other to grasp tissue therebetween;
a handle assembly at a proximal end of the elongate shaft, the handle assembly including:
a stationary handle;
a movable handle pivotally coupled to the stationary handle about a first pivot axis; and
a connector extending distally from the movable handle;
a drive assembly extending through the elongate shaft and operably coupled to at least one of the first and second jaw members;
a transmitter movable along the longitudinal axis and operably coupled to the drive assembly; and
a link having a first end pivotally coupled to the connector about a second pivot axis and a second end pivotally coupled to the transmitter about a third pivot axis, wherein pivotal movement of the movable handle toward the stationary handle causes the link to move the transmitter distally along the longitudinal axis to actuate the drive assembly and move the at least one of the first and second jaw members relative to the other.

16. The treatment system of claim 15, further comprising:
a rotation knob configured to rotate the first jaw member and the second jaw member about the longitudinal axis.

17. The treatment system of claim 15, further comprising:
one or more switches configured to receive treatment operation input from an operator.

18. The treatment system of claim 15, wherein the transmitter includes an engagement portion configured to engage with a slider of the drive assembly.

19. The treatment system of claim 15, wherein the link is arranged between a central axis along the longitudinal axis in the handle assembly and the first pivot axis.

20. The treatment system of claim 15, further comprising:
an ultrasound transducer configured to generate ultrasound vibration, wherein at least one of the first jaw member and the second jaw member is configured to transmit the ultrasound vibration to tissue grasped therebetween.
